(19) United States
(12) Patent Application Publication
(10) Pub. No.: US 2012/0197115 A1
(43) Pub. Date: Aug. 2, 2012

(54) SPECTRAL MAGNETIC PARTICLE IMAGING

(75) Inventors: Peter Leonardus Elisabeth Maria Pasmans, Eindhoven (NL); Denis Markov, Almere (NL)

(73) Assignee: KONINKLIJKE PHILIPS ELECTRONICS N.V., EINDHOVEN (NL)

(21) Appl. No.: 13/499,725

(22) PCT Filed: Oct. 11, 2010
(86) PCT No.: PCT/IB2010/054578

§ 371 Date: Apr. 2, 2012

(30) Foreign Application Priority Data
Oct. 15, 2009 (EP) 09173147.1

(51) Int. Cl. A61B 5/05; A61K 49/18; A61K 49/06

(52) U.S. Cl. 600/431; 424/9.3; 424/9.32

(57) ABSTRACT

The invention relates to the discrimination of spectroscopically different tracer materials by magnetic particle imaging (MPI), based on the differences of the MPI spectral responses of said tracer materials.

### FIELD OF THE INVENTION

The present invention refers to contrast agents capable of being detected by magnetic particle imaging (MPI), the use of said contrast agents as well as to a method for detecting said contrast agent by MPI.

### BACKGROUND OF THE INVENTION

Magnetic particle imaging (MPI) refers to a method to determine the spatial distribution of magnetic particles in an examination zone. Typically, MPI methods require the generation of a magnetic field with a spatial course of the magnetic field strength, which is such that a first partial region with a low magnetic field strength and a second partial region with a higher magnetic field strength are obtained in an examination area. The spatial position of the two partial regions in the examination area are changed so that the magnetization of the particles changes locally. The signals to be recorded are dependent on the magnetization in the examination area. The signals are finally evaluated so as to obtain information about the spatial distribution of the magnetic particles in the examination area.

For further explanations concerning MPI and the arrangement to be used for said method, reference is made to US 2003/0085703 A1.

### OBJECTS AND SUMMARY OF THE INVENTION

Known magnetic particle imaging techniques result in images that visualize the local concentration distribution of a tracer material in an object, but does not allow for discriminating between different types of tracer materials. Thus, currently magnetic particle imaging provides information (usually via grey scale images) about the concentration or distribution of one tracer material over the body tissue, but does not enable the discrimination between different tissue types or the detection of different functional areas via two or more tracer materials.

It is therefore an object of the present invention to provide means and method to overcome the aforementioned problem or limitation, to broaden the concept of MPI and/or to enable new diagnostic possibilities using MPI.

To address one or more of the above mentioned needs or objects, in a first aspect, the invention refers to a contrast agent comprising at least two different tracer materials suitable for magnetic particle imaging, wherein the tracer materials provide different MPI spectral responses and can be visualised or discriminated by MPI. The underlying concept allows for the dicrimination or differentiation of different tissue types, functional areas of in the body (e.g. organs) or any objects contrasted by the at least two tracer materials exhibiting different spectral MPI responses. In other words, the inventors of the present invention found that there is a possibility for detecting at the same time e.g. two different tracer materials by MPI techniques if said tracer materials provide sufficiently different MPI spectral responses.

The term “contrast agent” as used herein refers to a combination of at least two spectroscopically different tracer materials. The contrast agent according the present invention may represent one component containing at least two tracer materials or may be composed of at least two components each containing at least one tracer material. In other words, the contrast agent may e.g. be a tablet, suspension or any other device containing at least two tracer materials or may be a “kit” of at least two components (e.g. selected from tablets, suspensions, implants or any other device) each containing at least one tracer material. In the latter case, the components of the contrast agent may be delivered to the object to be examined or the targeting area in any possible order, including the simultaneous insertion of the two or more components.

The term “tracer material” as used herein refers to any material capable of being detected by MPI, including e.g. particlate materials or film or foil materials being applied to a medical device. It is to be understood that a tracer material also can be composed of several components. The term tracer material also encompasses modified and unmodified tracer materials.

The terms “spectroscopically different” and “different MPI spectral responses”, as used herein, are meant to reflect that the corresponding tracer materials are capable of being detected by MPI and that the at least two tracer materials provide different MPI spectral responses, wherein the difference between the MPI spectral responses is sufficient for visualising of or for discriminating between the particles.

According to one preferred embodiment of the invention, the at least two different tracer materials are in particulate form and the average particle size of the at least two different tracer materials differs by at least 10 nm. The “average particle size” in the meaning of the present invention refers to the primary particle size and may be determined by laser diffraction techniques or laser scattering. The particulate tracer materials preferably have a monodisperse particle size distribution.

According to another preferred embodiment, the MPI spectra of the at least one of the at least two spectroscopically different tracer materials shows at least 3 or at least 5 or at least 15 or at least 33 additional harmonics above the three times noise level in comparison to the MPI spectra of other tracer material of the at least two spectroscopically different tracer materials.

At least one of the at least two different tracer materials according to another preferred embodiment is contained in a medical device, which preferably is selected from the group consisting of implants, capsules, tablets and medical tools.

According to the present invention, it may be preferred that at least one of the at least two different tracer materials specifically localizes in or binds to a specific material, preferably a specific tissue (e.g. an organ tissue).

According to another embodiment of the present invention, at least one of the at least two different tracer materials is an iron oxide material, preferably a particulate iron oxide material.

Another aspect of the present invention is directed to the use of the inventive contrast agent for visualising of or discriminating between different materials or objects contrasted by the at least two tracer materials providing different MPI spectral responses.

According to a third aspect, the present invention relates to a method for discriminating between at least two different tracer materials by magnetic particle imaging (MPI), wherein the magnetic particle imaging process is carried out by detecting at least two tracer materials providing different MPI spectral responses.

According to an especially preferred embodiment of the inventive method, the method comprises at least the following steps:

(i) providing the at least two different tracer materials;
(ii) delivering the tracer materials to an object;
(iii) scanning the object for acquiring the signal Ŝ;
(iv) acquisition of a system function Ĝₓ for each tracer material X; and
(v) reconstruction of the image.

Preferably, step (v) involves the combination of the respective system functions Ĝₓ into a single matrix [Ĝ_{A} G_{B} . . . ] and this matrix is inverted to render [Ĝ_{A} Ĝ_{B} . . . ]⁻¹ e.g. by using Single Value Decomposition. Step (v) may also involve the determination of the local concentration distribution by using the following equation:

Ĉ=[Ĉ_{A} Ĉ_{B} . . . ]=Ŝ[Ĝ_{A} Ĝ_{B} . . . ]⁻¹,

wherein Ĉ is a matrix, which combines all different local concentration distributions Ĉ_{X} of the respective tracer material X detected in the object and Ĉ_{X} refers to the local concentration distribution of the respective tracer material X.

In still another aspect, the present invention refers to a computer readable medium having stored a program element, which, when being executed by a processing unit, is adapted to carry out anyone of the aformentiond methods.

These and other aspects of the invention will be apparent from and elucidated with reference to the embodiments described hereinafter.

### BRIEF DESCRIPTION OF THE DRAWINGS

FIG. 1 schematically shows the typical steps required for Magnetic Particle Imaging (MPI).

FIG. 2 schematically shows the inventive steps required for discriminating at least two spectroscopically different tracer materials by Magnetic Particle Imaging (MPI).

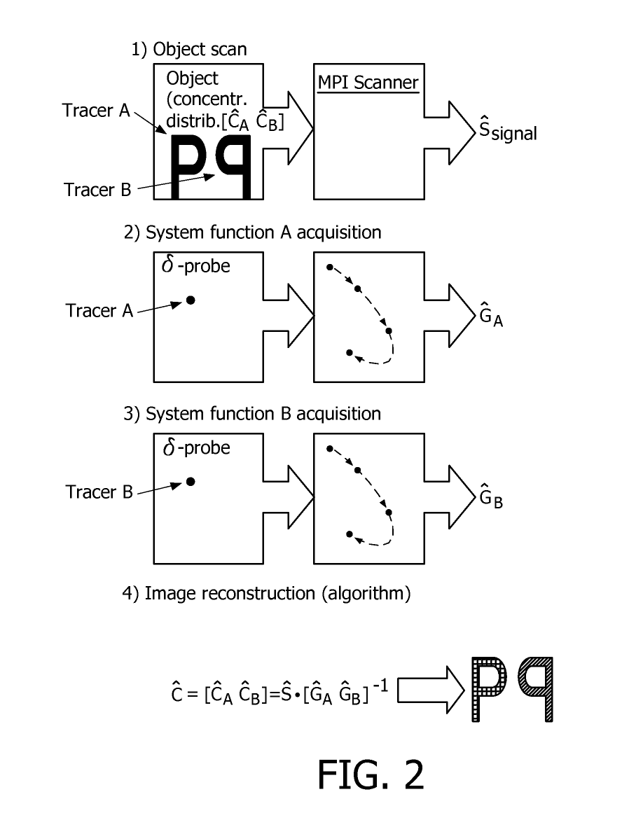

FIG. 3 shows a 2D-object and the simulated MPI image reconstructed according to the typical MPI method for scanning an object comprising one tracer material, wherein a) shows the 2D object (a plate with a number of drillings filled with a tracer material comprising 30 nm magnetic iron oxide particles) and b) shows the simulated MPI image.

FIG. 4 shows a 2D-object and the simulated MPI images reconstructed according to the inventive method, wherein a) shows the 2D object—a plate with a number of drillings filled with a tracer material comprising 30 nm magnetic iron oxide particles in the left drillings and 40 nm magnetic iron oxide nanomarticles in the right drillings, b) shows the simulated MPI image visualizing the 30 nm magnetic iron oxide particles (left P) together with the 40 nm magnetic iron oxide particles (right P).

### DETAILED DESCRIPTION OF THE EMBODIMENTS

According to one aspect of the present invention a contrast agent comprising at least two different tracer materials suitable for magnetic particle imaging (MPI) is provided, wherein the tracer materials show different MPI spectral responses such that the particles can be discriminated by the MPI techniques according to the present invention. It has been found that the signals generated by such contrast agents can be discriminated with regard to the spectroscopically different tracer materials, i.e. the signals obtained for the different tracer materials can be differentiated based on the different MPI spectral response. In other words, the inventors provide a method or concept which allow for the discrimination of MPI signals resulting from the corresponding tracer materials. By using the inventive contrast agent comprising at least two tracer materials, it is possible to obtain more information in a single measurement and to acquire more selective information.

The detection principle according to the present invention is based on the known MPI measuring or detection principle which involves the generation of a magnetic field with a spatial course of the magnetic field strength, which is such that a first partial region with a low magnetic field strength and a second partial region with a higher magnetic field strength are obtained in an examination area. In the first partial region, the magnetic field is so weak that the magnetization of the particles differs to a greater or lesser extent from the external magnetic field, that is to say is not saturated. In the second partial region (that is to say in the rest of the examination area outside the first part), the magnetic field is strong enough to keep the particles in a state of saturation. The magnetization is saturated when the magnetization of almost all the particles is oriented in approximately the direction of the external magnetic field, so that with any further increase in the magnetic field the magnetization increases to a much lesser extent there than in the first partial region leading to a corresponding increase in the magnetic field.

The first partial region is preferably a spatially coherent region; it may be a point-shaped region but may also be a line or a surface area. Depending on the configuration, the first partial region is spatially surrounded by the second partial region.

By changing the position of the two partial regions within the examination area, the (overall) magnetization in the examination area changes. The change in the spatial position of the partial regions may, for example, be effected by means of a temporally changing magnetic field. If the magnetization in the examination area or physical parameters influenced thereby are measured, information can be derived about the spatial distribution of the magnetic particles in the examination area.

To this end, for example, the signals induced in at least one coil due to the temporal change in the magnetization in the examination area are received and evaluated. If a temporally changing magnetic field acts on the examination area and on the particles in a first frequency band, then, of those signals received by the coil, only those signals which contain one or more higher frequency components than those of the first frequency band are evaluated. These measured signals are generated since the magnetization characteristic of the particles usually does not run in a linear manner.

Thus, the already known MPI imaging concept or the MPI procedure typically involves the following three steps:

1.) Object scan—acquisition of a signal Ŝ generated in the object across the filed of view (FOV)
2.) Reference scan—results in an acquisition of a system function Ĝ
3.) Image reconstruction—deriving of the actual tracer concentration distribution in the object.

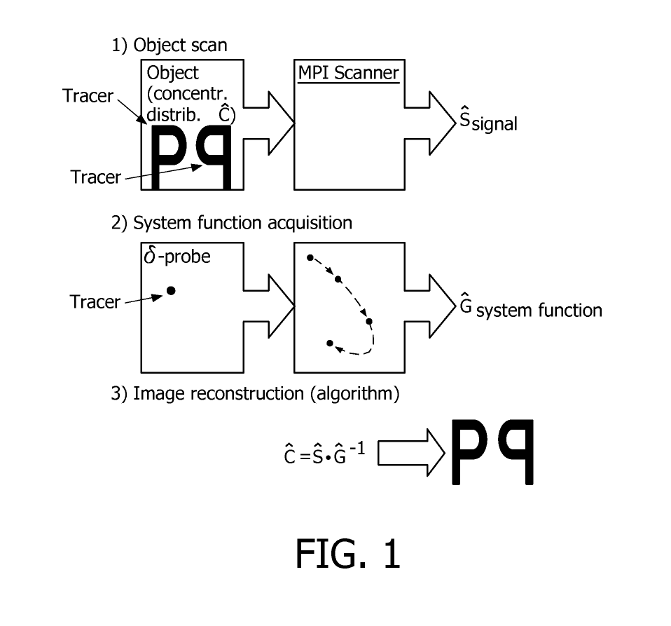

The foregoing steps are schematically illustrated in FIG. 1. The result of the aformentioned MPI method involving the scanning or measuring of a signal Ŝ for the tracer material in the object to be examined, the determination of the corresponding system function Ĝ and the image reconstruction by using the parameters Ĝ and Ŝ leads to high sensivity hot-spot grey scale images. The grey scale visually represents the quantitative information on local concentration of the magnetic tracer material. The evaluation of the acquired MPI signals is described in more detail in, for example, J. Weizenecker et al., Physics in Medicine and Biology 2007, vol. 52, pages 6363-6374. The corresponding methods may also be applied for the inventive methods, as far as applicable.

According to the present invention, a method is provided which allows for the use of the inventive contrast agents comprising at least two spectroscopically different tracer materials. The inventive method for visualizing at least two different tracer materials by magnetic particle imaging, is carried out by detecting at least two tracer materials providing different MPI spectral responses. The inventive method may comprise at least the following steps:

(i) providing the at least two different tracer materials;
(ii) delivering the tracer materials to an object;
(iii) scanning the object for obtaining the signal Ŝ;
(iv) acquisition of a system function Ĝ for each tracer material; and
(v) reconstruction of the image.

It is to be understood that these steps can be carried out in any order, unless the context clearly dictates otherwise, and the actions comprised in one step do not have to be carried out without interruption. For example, the acquisition of the system function Ĝ might be performed anytime before the object scan and the acquisition of the remaining system functions might be performed anytime after the object scan. This method makes it possible to discriminate between multiple spectroscopically different tracer materials. More precisely, the inventive method renders it possible to determine the local concentration distributions of at least two spectroscopically different tracer materials. In the following, reference is made to the materials suitable for the inventive method and the inventive contrast agents as well as the technical details and the parameters which may be used for the present invention.

### Steps (i) and (ii):

As set out above, the present invention is based on the finding that spectroscopically different tracer materials can be visualized or discriminated via MPI. The at least two tracer materials according to the present invention are part of the inventive contrast agent. In the following, reference will be made to the tracer materials to be provided according to step (i). The tracer materials according to step (ii) are to be delivered to the object of interest.

One specific embodiment of the invention refers to the combination of at least two spectroscopically different tracer materials that are detected within the object or specific parts of the object, e.g. the human body or parts thereof. Based on the findings according to the present invention, it is possible to provide a modular system of contrast agent components comprising specific spectroscopically different tracer materials that can be combined as required for the specific MPI procedure or e.g. the diagnostic purposes.

Thus, one embodiment of the present invention refers to a contrast agent, wherein at least two spectroscopically different tracer materials differ with respect to their modification. This renders it possible to achieve selectivities with regard to the distribution of the tracer materials within the object to be examined, e.g. the human or animal body.

For example, one or more of the at least two spectroscopically different tracer materials may form, be bound to or localized at a medical device or a specific region of the human or animal body during measurement. The tracer material may be fixed to such medical device during measurement or may be released from it over time before, during and/or after measurement. Alternatively, the medical device may be made form the tracer material material. The release from such a device can be based on diffusion, solution or other mechanisms, wherein the release can be independent or almost independent from outside stimuli or the environment or, alternatively, could be controlled by one or more outside stimuli, like a specific temperature or pH-value. According to one inventive embodiment, one or more of the inventive tracer materials forming the contrast agent are fixed to a device, especially a medical device if the object is a human or animal body or a part of a human or animal body. The term “medical device” as used herein refers to a device used in medicine, for example, a capsule, implant or medical tool. The term “implant” as used herein, refers to a medical device that is implanted in the human or animal body. The term implant comprises, but is not limited to, vascular endoprostheses, intraluminal endoprostheses, stents such as coronary stents or peripheral stents, surgical, dental or orthopedic implants, implantable orthopedic fixation aids, orthopedic bone prostheses or joint prostheses, artificial hearts or parts thereof, artificial heart valves, heart pacemaker casings or electrodes, subcutaneous and/or intramuscular implants, implantable drug-delivery devices, microchips, or implantable surgical needles, screws, nails, clips, or staples, or seed implants or the like. Typically, implants are made of solid materials, either polymers, ceramics or metals. To enable the delivery or release of tracer materials, implants can also be produced with porous surfaces or by using porous materials, wherein a tracer material may be included in the pore system for in vivo release.

One embodiment of the present invention refers to a contrast agent, wherein at least one of the spectroscopically different tracer materials comprises one or more coating(s) and the surface of the outer coating is functionalized. Modifying at least one of the spectroscopically different tracer materials may make it possible that the spectroscopically different tracer materials can be used in specific environments and/or that the spectroscopically different tracer materials provide specific selectivities with regard to the materials the tracer materials localize in or bind to.

In one embodiment of the present invention the contrast agent comprises at least two spectroscopically different tracer materials, wherein these tracer materials address spatially different locations of interest. This renders it possible to discriminate between the spectroscopically different tracer materials more easily, as the signals originating from multiple tracer materials do not or at least almost not overlap.

In another embodiment, wherein the tracer materials are in particulate form, the particle size may be in the range of 1 to 10,000, 1 to 400, 1 to 50, 3 to 2000, 3 to 500, 10 to 2000, 10 to 500, 10 to 50, 15 to 2000, 15 to 100, 15 to 50, 20 to 400, 20 to 2000, 20 to 100 or 20 to 50 nm. It is to be understood that the above mentioned ranges are only specific embodiments and the person skilled in the art is able to choose other, additional or more specific ranges for the use in a specific system, based on the present invention combined with general knowledge of the specific system, for example the specific physical and/or chemical properties of a scanned object. A contrast agent comprising tracer materials that disperse in the object can be, for example, limited by the physical properties of the system. If such particles, for example, should disperse in the blood stream of a human body, the overall size of those particles is limited by the smallest diameter of the capillaries they have to pass. Therefore, the overall diameter of such particles may be preferably below 2 μm, more preferred below 1.5 μm and even more preferred below 1 μm, if the particles should disperse in the human body by the blood stream.

Specific embodiments refer to combinations of spectroscopically different tracer materials providing a significantly different magnetic response which may facilitate discrimination of the tracer materials. It is indicated that it is measurable whether two tracer materials are “spectroscopically different” by a) aquiring MPI spectra for the respective tracer materials or materials (A and B), b) normalizing the obtained spectra by the 3d harmonic of the base frequency, and c) validating if there are additional multiple harmonics above 3 times the noise level in case of the tracer B (A). Preferably, the MPI spectra of the at least one of the at least two spectroscopically different tracer materials shows at least 3 or at least 5 or at least 15 or at least 33 additional harmonics above the three times noise level in comparison to the MPI spectra of other tracer material of the at least two spectroscopically different tracer materials. Preferably, the corresponding signals should have a 10% difference in signal intensity of any harmonics.

Another embodiment of the present invention refers to a contrast agent comprising at least two spectroscopically different tracer materials, wherein the difference of the particle size of the at least two spectroscopically different tracer materials is at least 1 nm, at least 2 nm, at least 3 nm, at least 4 nm, at least 5 nm, at least 6 nm, at least 7 nm, at least 8 nm, at least 9 nm, at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 21 nm, at least 22 nm, at least 23 nm, at least 24 nm, at least 25 nm, at least 26 nm, at least 27 nm, at least 28 nm, at least 29 nm or at least 30 nm, if the corresponding spectroscopically different tracer materials are particles. The inventors found that the required difference of the MPI spectral response can be achieved by using the particulate tracer materials of different size.

The aforementioned embodiments can be used individually or combined to create a preferred embodiment.

The tracer materials may be composed of any suitable material known to the person skilled in the art. The tracer material may be composed of a magnetic material, preferably of Fe, Co, Ni, Zn, Mn etc. or chemical derivatives thereof. Typical derivatives which are preferable envisaged by the present invention are alloys or oxides of metals, e.g. alloys or oxides of Fe, Co, Ni, Zn or Mn or any combination thereof. Particularly preferred are oxides of Fe, generally specified as FeₓO_{y}, e.g. Fe₂O₃ or Fe₃O₄. Also encompassed by the present invention are tracer materials composed of ferrite material or doped material, e.g. Co, Ni, Zn or Mn:FeₓO_{y}. Suitable tracer materials include the commercially available Resovist and Endorem.

Preferably the magnetic material comprised in the tracer material comprises only a single magnetic domain and/or no Weiss zones.

In one embodiment at least one of the spectroscopically different tracer materials is a tracer material that is capable of being detected by MPI only if localized in or bound to a specific material. In another embodiment at least one of the spectroscopically different tracer materials is a tracer material that is capable of being well detected by MPI, if specific physical and/or chemical requirements are fulfilled. An example for a specific physical requirement is a specific temperature. Examples of specific chemical requirements are specific pH-values or the presence of a specific compounds. This renders it possible to detect a specific material or detect a specific area of interest that fulfills the corresponding requirement.

The tracer materials to be used according to the present invention can be modified in various ways. This makes it possible to adapt the properties of the tracer material to specific requirements. For example, a coating could provide resistance against a specific environment. For example, a pharmaceutically acceptable shell could prohibit side effects that would occur using unmodified tracer materials in the human or animal body. For example, a functionalization can result in a specifically localizing in or binding of the modified tracer material to a specific material, like a specific tissue in the human body.

One inventive embodiment refers to tracer materials having a coating region at least partly enclosing the surface. The coating may be biocompatible, i.e. biodegradable and/or biostable, so that particle cluster formation is prevented by a combination of different forces including electrostatic repulsion from ionic charges in the coating or steric hindrance. As a result, colloidal stability can be sustained during tracer material fabrication, storage and use. In a further alternative embodiment, it may be possible to gather information on the environment of the magnetic particles during detection. Especially, it is, e.g., possible to provide the coating region such that the coating region removes from the particle if a predefined temperature in the environment of the magnetic particles is exceeded. The term “biocompatible” as used herein refers to the feature that the corresponding labelled material does not cause toxic or injurioues effects on biological system, based on the intended use. The term “biodegradable” as used herein refers to the feature that a substance can be cleaved into small units that can be used by the body and/or can be removed by the kidneys. Examples of biodegradable organic materials are natural or synthetic polymers, e.g., collagen, cellulose, silicone, poly(alpha esters) such as poly(lactate acid), poly(glycolic acid), polyorthoesters or polyanhydrides.

In an exemplary embodiment the biodegradable material comprises a hydrogel. Hydrogels can be made biodegradable with a wide range of degradation times which may be useful, e.g., in tissue engineering, cell therapy applications or controlled release of pharmaceutically active agents. Examples of biodegradable inorganic materials are metals or alloys based on at least one of magnesium or zinc, or alloys comprising at least one of Mg, Ca, Fe, Zn, Al, W, Ln, Si, or Y. Also suitable are, e.g., alkaline earth metal oxides or hydroxides, such as magnesium oxide, magnesium hydroxide, calcium oxide, and calcium hydroxide or mixtures thereof. In an exemplary embodiment of the invention, the biocompatible product can also comprise inorganic composites or organic composites or hybrid inorganic/organic composites.

Specific embodiments refer to the modification of the pharmacological properties, especially using a pharmaceutically acceptable shell to prohibit, for example, side effect occuring with the unmodified tracer material. Examples of pharmaceutically acceptable shells comprise a synthetical polymer or copolymer, a starch or a derivative, a dextran or a derivative, a cyclodextran or a derivative, a fatty acid, a polysasccharide, a lecithin, a mono-, di- or triglyceride or a derivative, a cell encapsulation or a liposomal encapsulation, especially a cell encapsulation or a liposomal encapsulation.

It is to be understood that the terms “coating” and “pharmaceutical acceptable shell” in specific cases are interchangable. Specific embodiments refer to tracer materials that are functionalized, for example, with one targeting ligand reactive to a target molecule or to a plurality of target molecules in an examination area. The tracer materials may preferably have a reduced rotational mobility after binding to the target molecule or target molecules, wherein the at least one targeting ligand is preferably a biological entity, especially an amino acid or polypeptide or a nucleic acid, and wherein the target molecule is preferably a biological entity, especially an enzyme or a nucleic acid or an antibody. Such an functionalization could be used to achieve, for example a selective binding of the tracer material to plaque, like vulnerable plaque or to a specific organ in the human or animal body. Another example would be a functionalization that results in a specifically localizing in or binding to an implant. Within the context of the present invention, a “ligand” is a substance which binds to a given substance with an IC₅₀ of less than 10 μM, preferably of less than 1 μM, less than 500 nM, less than 200 nM, less than 100 nM, less than 30 nM or less than 20 nM. In the prior art, a plurality of methods are known for determining the binding affinity of a ligand (IC₅₀ or some other parameter) to a given substance. These methods include, without limitation, ELISA, surface plasmon resonance and radio ligand binding assay methods, as described for example in Gazal S. et al, J. Med. Chem. 2002, 45: 1665-1671. Other functionalizations might primarily influence the more generic parameters of the tracer material, like the hydrophilic, lipophilic, hydrophobic and/or lipophobic properties.

It is to be understood that further possible modifications are known to the person skilled in the art and that all modifications can be applied in combination for the purpose of the present invention.

Steps (iii) and (iv):

The steps of (iii) scanning the object for acquiring the signal Ŝ and (iv) acquisition of a system function Ĝ for each tracer material will be decribed in the following in more detail. Reference is also made to details and explanations set out above with respect to the standard MPI procedure. For carrying out the inventive method for discriminating between tracer materials providing spectroscopically different magnet responses or using the iventive contrast agents, standard MPI equipment as describe above may be used. In other words, the signal Ŝ can be measured or detected according to the known standard procedure. The term “Ŝ” as used herein refers to the signal acquisition, resulted from the local concentration distribution of the tracer materials across the field of view. Beside the aformentioned object scan according to step (iii), which involves the scanning of the object containing the inventive contrast agent, i.e. at least two tracer materials providing different magnetic responses, a “reference scan” is to be carried out according to step (iv). According to the inventive method, said reference scan is to be carried out for each tracer material used in step (iii). Generally speaking, the reference scan results in an acquisition of a system function Ĝ. It is well known to the skilled person how to obtain said Ĝ. A system function Ĝ represents the MPI scanner response to a point-like confinement of tracer materials moved in a step-like manner across the field of view. According to the present invention, several system functions Ĝₓ are to be determined. More precisely, for each tracer material used in step (iii) one system function Ĝₓ must be aquired. The corresponding reference scans can be carried out one after the other for the respective tracer material. The term “Ĝₓ” as used herein refers to a specific system function, wherein X relates to the corresponding material. The system function denotes the delta response of the MPI scanner system, representing the signal induced (in frequency domain) in the scanner as a result of a point-like object filled with a tracer material X moving across the field-of-view.

### Step (v):

In step (v) of the inventive method, the image reconstruction is carried out. In order to generate the image reconstruction according to the method of the present invention, the system functions Ĝₓ obtained in step (iv) are combined into a single matrix [Ĝ_{A} Ĝ_{B} . . . ]. This matrix according to the present invention is inverted, preferably using Single Value Decomposition. The specific local concentration distribution of the tracer materials can be determined or derived from the inverted matrix according to the following equation:

Ĉ=[Ĉ_{A}Ĉ_{B} . . . ]=Ŝ[Ĝ_{A}Ĝ_{B} . . . ]⁻¹

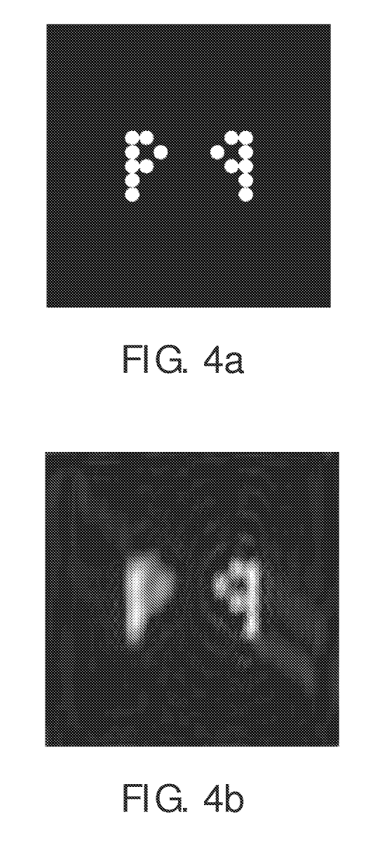

The term “Ĉhd x” as used herein refers to the local concentration distribution, wherein X relates to the corresponding material. The term “Ĉ” as used herein refers to the matrix, which combines all different tracer concentration distributions. The resulting reconstructed images may be grey scale images or may be displayed as coloured images. The grey scale or colour scale visually represents the quantitative information on local concentration of the magnetic tracer material. Examples for corresponding images are shown in FIG. 4.

The skilled person knows how to adjust suitable image acquisition and reconstruction parameters like Field Of View (FOV), field gradient, field amplitude and regularization parameter λ for simulations with or without noise models. The system functions for two contrast materials may, for example, be 100×100 with Field Of View (FOV) of 20×20 mm. The harmonics used for reconstruction may be 5000 (2500 per coil). Filtering may be based on highest average intensity of system functions. A suitable field gradient in the horizontal direction may be 2.5 T/m. The field amplitude used may be 20 mT. λ may be in the range of 10⁻²² to 10⁻²⁶.

The inventive concept provided by the inventors allows for measurements and diagnostic procedures which cannot be realized with conventional MPI procedures using only one tracer material. Since the tracer materials according to the present invention can be used to detect and locate specific materials with a particularly high spatial resolution, they are used in one embodiment for the diagnosis of proliferative diseases, and in particular early phases of such diseases. Proliferative diseases include e.g. a tumor and a precancerous condition.

Further diseases which can be diagnosed according to the present invention include autoimmune diseases which are selected from the group consisting of rheumatoid arthritis, inflammatory bowel disease, osteoarthritis, neuropathic pain, alopecia areata, psoriasis, psoriatic arthritis, acute pancreatitis, allograft rejection, allergies, allergic inflammation in the lungs, multiple sclerosis, Alzheimer's disease, Crohn's disease, and systemic lupus erythematosus.

One embodiment of the present invention refers to a method to determine the local concentration distribution of at least one of the spectroscopically different tracer materials in a scanned object comprising at least two spectroscopically different tracer materials using MPI, based on the differences in the MPI spectral responses. This renders it possible to examine the scanned object with regard to the distribution of specific tracer materials. Another embodiment refers to a method, wherein the method comprises the determination of a local concentration distribution that does not comprise the corresponding local concentration distribution with regard to at least one of the spectroscopically different tracer materials.

In another embodiment of the present invention the contrast agent comprises at least two spectroscopically different tracer materials that disperse in the object, wherein at least one spectrocopical different tracer materials specifically localizes in or binds to a specific material and at least one of the spectrocopical different tracer materials can be used as point of reference based on its distribution. This renders it possible to detect a specific material, preferably a specific tissue, especially pathological tissue, while simultaneously locating it. This could be used, for example, to locate plaque, like vulnerable plaque, by combining a tracer material that binds to plaque, like vulnerable plaque, and another tracer material that remains in the blood stream. As set out above, the tracer materials contained in the inventive contrast agent can be used for “active” and “passive” targeting, i.e. the tracer materials can be modified e.g. by a coating so that it specifically (actively) binds to a target region or could be used unmodified for passive targeting.

In still another embodiment the contrast agent comprises at least two spectroscopically different tracer materials, wherein at least one spectroscopically different tracer material disperses in the object and at least one spectroscopically different tracer material is fixed to a device or forms the device, especially a medical device. This makes it possible, for example, to locate the position of medical device, like a capsule, an implant or a medical tool, and the information obtained by MPI could therefore be used for non-invasive interventions. For example, by combining an implant, like a catheter, comprising at least one of the spectroscopically different tracer material and at least one spectroscopically different tracer material that localizes in or binds to the surrounding and/or neighbouring material, like the circulating blood.

The inventive method not only allows for the diagnosis of cancer and cardio-vascular (CV) diseases, but also for the visualization of catheters, stents and other artificial constructs and devices.

According to a further embodiment of the present invention, a computer readable medium having stored a program element is provided, which, when being executed by a processing unit, is adapted to carry out anyone of the inventive methods described herein. The present invention furthermore refers to computer programs to carry out the aforementioned method and/or its embodiments. Especially it refers to computer readable medium having stored a program element, which, when being executed by a processing unit, is adapted to carry out the aforementioned methods.

Wherein an indefinite of definite article is used in conjunction with a singular noun, for example “a”, “an”, “the”, this includes a plural of that noun unless specifically stated otherwise.

The term “comprising” as used herein should not be interpreted as being restricted to the means listed thereafter, it does not exclude other elements or steps.

It has to be understood that one specific embodiment, a combination of multiple embodiments or even a combination of all embodiments described herein may be selected to achieve a specific preferred embodiment, unless it is clear that specific embodiments can not be combined. In this case the person skilled may use any of the mutual exclusive embodiments, wherein each of these especially preferred embodiments may be selected. Furthermore, it is to be understood, that the mere fact that certain measures are recited in mutually different dependant claims does not indicate that a combination of these measures cannot be used to advantage.

It is further to be understood that while the present invention has been described in detail with respect to specific embodiments thereof, it should be noted that the above-mentioned embodiments are to be considered illustrative or exemplary and not restrictive; the invention is not limited to the disclosed embodiments. Variations to the disclosed embodiments can be understood and effected by those skilled in the art in practicing the claimed invention, from a study of the disclosure, and the appended claims.

### EXAMPLES

### Comparative Example

### Simulation Study Using 30 Nm Magnetic Iron Oxide Particles to Determine the Local Concentration Distribution Magnetic Iron Oxide Particles

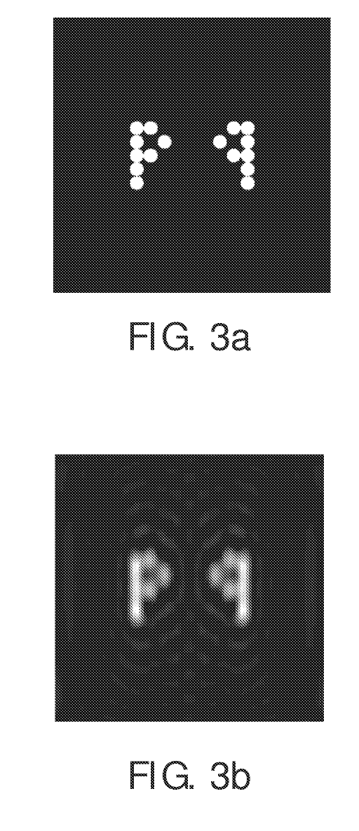

In this image simulation study a typical MPI method using one tracer material is used to determine the local concentration distribution of 30 nm magnetic iron oxide particles. The 2D-object and the reconstructed MPI image are shown in FIG. 3. The simulation parameters are:

Object: each character (‘P’s) is formed by 8 cylinders with diameters of 1 mm
Simulation is carried out without a noise in the model
System functions: 100×100 with Field Of View (FOV) of 20×20 mm
Harmonics used for reconstruction: 5000 (2500 per coil)
(filtering based on highest average intensity of system functions)
Field gradient: 2.5 T/m (horizontal direction)
Field amplitude: 20 mT
λ=10-25.

### Example 1

### Simulation Study Using 30 Nm and 40 Nm Magnetic Iron Oxide Particles to Determine the Local Concentration Distribution Magnetic Iron Oxide Particles

In this image simulation study, two types of particles are included: particles described by the Langevin function with diameters of 30 nm and 40 nm, wherein the acquired data was processed using the inventive method. The 2D-object and the reconstructed MPI images are shown on FIG. 4. The local concentration distributions and system functions are labelled according to the aformentioned specification, wherein the indices 30 and 40 represent the size of the corresponding particles. The Signal Ŝ can be calculated using

Ĉ₃₀Ĝ₃₀+Ĉ₄₀Ĝ₄₀=[Ĉ₃₀Ĉ₄₀] [Ĝ₃₀Ĝ₄₀]=Ŝ,

where [. . . ] stands for concatenation along the direction of the positions i. For the system functions discrete positions i=100×100 are used. The number of spectral components taken into account are 5000 (with the same components for both matrices), whereby the matrices Ĝ₃₀ and Ĝ₄₀ have a size of 10000×5000 and the single matrix [Ĝ₃₀ Ĝ₄₀] has thus a size of 20000×5000 and the length of Ŝ is 5000.
Reconstruction of [Ĉ₃₀ Ĉ₄₀] (a vector with length 20000) is performed by Ŝ[Ĝ₃₀ Ĝ₄₀]⁻¹=[Ĉ₃₀Ĉ₄₀],
wherein the inversion is performed using the SVD.
The simulation parameters are identical to the comparative Example.

1. A contrast agent comprising at least two different tracer materials suitable for magnetic particle imaging (MPI), wherein the tracer materials provide different MPI spectral responses.

2. The contrast agent according to claim 1, wherein at least one of the at least two different tracer materials is contained in a medical device.

3. The contrast agent of claim 2, wherein the medical device is selected from the group consisting of implants, capsules, tablets and medical tools.

4. The contrast agent according to claim 1, wherein at least one of the at least two different tracer materials specifically localizes in or binds to a specific material, preferably a specific tissue.

5. The contrast agent according to claim 1, wherein the at least two different tracer materials are in particulate form and the average particle size of the at least two different tracer materials differs by at least 10 nm.

6. The contrast agent according to claim 1, wherein the MPI spectra of the at least one of the at least two spectroscopically different tracer materials shows at least 3 or at least 5 or at least 15 or at least 33 additional harmonics above the three times noise level in comparison to the MPI spectra of other tracer material of the at least two spectroscopically different tracer materials.

7. The contrast agent according to claim 1, wherein at least one of the at least two different tracer materials is an iron oxide material, preferably a particulate iron oxide material.

8. Use of a contrast agent according to claim 1 for visualizing of or discriminating between different materials or objects contrasted by the at least two tracer materials providing different MPI spectral responses.

9. Method of discriminating between at least two different tracer materials by magnetic particle imaging (MPI), wherein the magnetic particle imaging process is carried out by utilizing at least two tracer materials providing different MPI spectral responses.

10. The method of claim 9, wherein the method comprises at least the following steps:
(i) providing the at least two different tracer materials;
(ii) delivering the tracer materials to an object;
(iii) scanning the object for acquiring the signal Ŝ;
(iv) acquisition of a system function Ĝₓ for each tracer material X; and
(v) reconstruction of the image.

11. The method of claim 10, wherein step (v) involves the combination of the respective system functions Ĝₓ into a single matrix [Ĝ_{A} Ĝ_{B} . . . ] and this matrix is inverted to render [Ĝ_{A} G_{B} . . . ]⁻¹.

12. The method of claim 10, wherein step (v) involves the determination of the local concentration distribution by using the following equation:
Ĉ=[Ĉ_{A} Ĉ_{B} . . . ]=Ŝ[Ĝ_{A} Ĝ_{B} . . . ]⁻¹,
wherein Ĉ is a matrix, which combines all different local concentration distributions Ĉₓ of the respective tracer material X detected in the object.

13. The method of claim 9, wherein the at least two different tracer materials are comprised in a contrast agent.

14. A computer readable medium having stored a program element, which, when being executed by a processing unit, is adapted to carry out the method of claim 9.
